(19) United States
(12) Patent Application Publication
(10) Pub. No.: US 2004/0171923 A1
(43) Pub. Date: Sep. 2, 2004

(54) Devices, systems and methods for improving vessel access

(76) Inventors: John F. Kalafut, Oakmont, PA (US); Arthur E. Uber III, Pittsburgh, PA (US); Edward J. Rhinehart, Monroeville, PA (US); Jill C. Uhryniak, Fox Chapel, PA (US); Donald M. DeLauder, Wexford, PA (US); David M. Reilly, Glenshaw, PA (US)

Correspondence Address: GREGORY L BRADLEY, MEDRAD INC, ONE MEDRAD DRIVE, INDIANOLA, PA, 15051

(21) Appl. No.: 10/729,434

(22) Filed: Dec. 6, 2003

Related U.S. Application Data
(60) Provisional application No. 60/431,289, filed on Dec. 6, 2002.

(51) Int. Cl. A61B 5/05

(52) U.S. Cl. 600/407

(57) ABSTRACT

An apparatus and method for use in accessing a vasculature of a patient includes a pedestal, a mobility mechanism, a limb support table, at least one storage device, and a vessel visualization device. Operably associated with the pedestal, the mobility mechanism enables the apparatus to be mobile. The limb support table is also operably associated with the pedestal and is used to support a limb of the patient. The at least one storage device is operably associated with the pedestal and/or the limb support table. The vessel visualization device is operably associated with the pedestal and/or the limb support table and is used for improving visualization of the vasculature of the patient.

### CROSS-REFERENCE TO RELATED APPLICATION(S)

This application for patent claims the benefit of U.S. Provisional Application Serial No. 60/431,289 titled Devices, Systems And Methods For Improving Venous Access, filed Dec. 6, 2002. This provisional application has been assigned to the assignee of the invention disclosed below, and its teachings are incorporated into this document by reference.

### FIELD OF THE INVENTION

The invention generally relates to devices used to gain access to the vasculature of the patient. More particularly, the invention pertains to devices, systems and methods for assisting medical personnel to access the vessel(s) of a patient more quickly and efficiently than is possible with prior art devices, systems and methods.

### BACKGROUND OF THE INVENTION

Gaining access to the vessel of a patient is often a necessary part of a medical diagnosis or treatment. One example of this is drawing blood for laboratory analysis. Access to a vein is also commonly performed for intravenous (IV) delivery of fluids or drugs. In some imaging procedures, drugs or contrast media (contrast) are administered to help the doctor modify or visualize the patient's condition. If this IV access is performed in the imaging suite, the time required to gain access to the patient's vein takes away from the time the imager is in use, and, if prolonged, can delay other patients. When patients are significantly ill, obese, or have veins damaged by cancer drugs or repeated accesses, it can take tens of minutes and multiple attempts by several hospital personnel to successfully place the IV needle or catheter. This increases the morbidity for the patient and cost to the medical institution.

Many types of medical practitioners may need to access the vasculature of a patient. A phlebotomist's full time job is to draw blood for laboratory tests. Doctors are often called upon for especially difficult cases. Nurses and imaging technologists often place IVs for medical imaging procedures. Many companies, universities, and medical facilities have made available on the Internet written guidelines or procedures for achieving IV access. One excellent guide is “Essentials of IV Therapy,” published by Baxter Healthcare Corporation, the contents of which are incorporated herein by reference.

To gain IV access, a hollow needle is typically inserted through the skin and into the vessel of the patient. This is sufficient for access durations on the order of seconds to a few minutes. For longer durations or if the patient will be moving the limb, it is preferred to have a catheter in the vessel and to remove the needle. There are both “needle over catheter” and “catheter over needle” systems manufactured by many companies. One example is the Jelco IV catheter made by Johnson & Johnson of 2500 East Arbrook Blvd., Arlington, Tex. 76014. Also, the Intima catheter has additional safety features and is manufactured by Becton Dickinson, Deseret Medical, of Sandy, Utah 84070.

There are many products that have been designed to help hospital personnel gain IV access. For example, there are phlebotomy chairs, with and without supply storage drawers, in which the patient sits so that their arms are supported for IV placement. Custom Comfort, Inc. of Orlando, Fla. 32867-7189, and Pinestar Technology of Greenville, Pa. 16125, are two manufacturers of these types of chairs. There are phlebotomy trays that nurses can use to carry supplies to patients' rooms. There are specific products, such as the MySono, a small ultrasound scanner made by Medison Co., Ltd in Korea, the Sonic Flashlight listed on the Internet as having patents pending and under development by George Stetten at Carnegie Mellon University, or infrared imagers (e.g., those in U.S. Pat. Nos. 4,817,622, 5,519,208, and 6,230,046) that can assist the nurse or doctor in seeing the veins of a patient.

When gaining venous access for injection of contrast during a CT or MRI imaging procedure, the process is further complicated by the fact that the patient is lying on a couch or bed that is movable to position the patient inside the gantry of the scanner. There is often little room to hold the necessary disposable supplies. Often the attending nurse puts them on the patient's abdomen. There is often no place to support the patient's arm. Some technicians support the patient's arm by holding the patient's wrist between their arm and body. Not surprisingly, it is difficult to remove the needle and quickly put the cap on the IV catheter so the patient's blood does not escape, and then tape the IV catheter to the arm to secure it.

In addition, for CT procedures where a bodily region below the neck is being imaged, the arms need to be placed over the patient's head so that they are not in the image, which would cause scatter that can degrade the image. However, when a patient puts his/her arms over their head, if they bend them too far, the flow of contrast or medication through the vein into the body can be hindered, causing an unsatisfactory image or increasing the possibility of an extravasation. The injection flow rates during a CT imaging procedure can be up to 5 to 7 ml/second, which requires good, uncompromised venous flow.

### SUMMARY OF THE INVENTION

The objectives and advantages of the invention are attained by the various embodiments and related aspects of the invention summarized below.

In a presently preferred embodiment, the invention provides an apparatus for use in accessing a vasculature of a patient. The apparatus comprises a pedestal, a mobility means, a limb support table, at least one storage device, and a vessel visualization device. Operably associated with the pedestal, the mobility means enables the pedestal be mobile. The limb support table is also operably associated with the pedestal and is used to support a limb of the patient. The at least one storage device is operably associated with the pedestal and/or the limb support table. The vessel visualization device is operably associated with the pedestal and/or the limb support table and is used for improving visualization of the vasculature of the patient.

In a broader embodiment, the invention provides an apparatus for use in accessing a vasculature of a patient. The apparatus comprises a limb support table and a mounting system. The limb support table is used for supporting a limb of the patient. The mounting system is used for mounting the limb support table in proximity to and adjustably with respect to the patient. In one aspect, the mounting system may take the form of an overhead counterpoise by which the limb support table is mountable in proximity to the patient. In another aspect, the mounting system may be manifested at least in part as a first articulating arm by which the limb support table is mountable to a table for the patient or a dedicated chair for the patient. In yet another aspect, the mounting system may include a mobile pedestal to which the limb support table is adjustably mounted and thus made mobile.

The invention also provides a method of accessing the vasculature of the patient, which uses the aforementioned apparatus according to, for example, its presently preferred embodiment. The method further comprises the steps of: moving the apparatus in proximity to the patient; placing the limb of the patient on the limb support table; retrieving an access conduit from the at least one storage device; inserting the access conduit into a vessel of the patient; strain relieving the access conduit; and removing the limb of the patient from the limb support table.

### BRIEF DESCRIPTION OF THE DRAWINGS

The invention, and particularly its presently preferred and alternative embodiments and related aspects, will be better understood by reference to the detailed disclosure below and to the accompanying drawings, in which:

FIG. 1 illustrates an IV assist apparatus according to a generalized embodiment of the invention;

FIG. 2 illustrates an IV assist apparatus according to a first presently preferred embodiment of the invention, with several parts functionally similar to those shown in FIG. 1;

FIG. 3 illustrates an IV assist apparatus according to a second presently preferred embodiment of the invention, with several parts functionally similar to those shown in FIGS. 1 and 2;

FIG. 4 illustrates the IV assist apparatus of FIG. 3 in relation to a medical imager; and

FIG. 5 illustrates an IV assist apparatus according to a third presently preferred embodiment, which is mounted in cooperation with a medical imager.

### DETAILED DESCRIPTION OF THE INVENTION

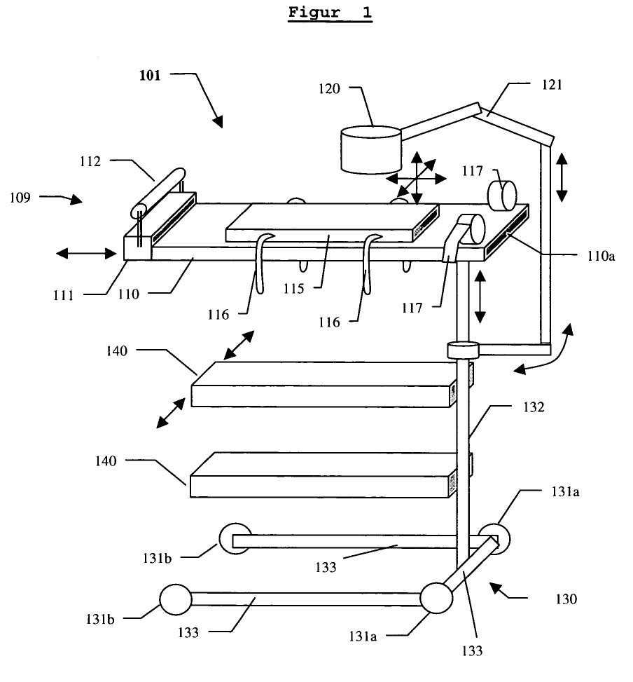

FIG. 1 shows an IV assist apparatus, generally designated 101, according to a generalized embodiment of the invention. It is intended to assist a nurse or other qualified personnel in accessing the vasculature of a patient. The IV assist apparatus 101 includes a pedestal 130, a mobility means 131, a limb support table 109, and a vessel visualization device 120. It also preferably includes at least one storage device 140. Operably associated with pedestal 130, the mobility means 131 enables the pedestal be mobile. The limb support table 109 is also operably associated with pedestal 130 and is used to support a limb of the patient. The storage device(s) 140 are operably associated with pedestal 130 and/or the limb support table 109. Used for improving visualization of the vasculature of the patient, the vessel visualization device 120 is operably associated with pedestal 130 and/or the limb support table 109.

The pedestal 130 preferably includes a base 133 and a structural member 132. As shown in FIG. 1, the structural member 132 can take the form of a pole to which the limb support table 109 is securable and preferably adjustable in relation thereto. The mobility means 131 can be manifested, for example, as a plurality of wheels 131a and 131b or one or more glides (not shown) attached to the base 133 of pedestal 130. It could also take the form of an air cushion system (not shown).

Whether manifested as wheels, glides or an air cushion, the mobility means 131 allows IV assist apparatus 101 to be moved to whatever position is necessary with respect to the patient who is, for example, already positioned on the table of a medical imager or in a chair, a wheelchair, or a bed. In FIG. 1, the IV assist apparatus 101 is shown with four wheels, all of which may be pivotable. Alternatively, for example, wheels 131b could pivot while wheels 131a do not. Other manifestations of the mobility means 131 may be preferable in different situations. For example, three wheels could be used if the floor is uneven. The most preferable mobility means in terms of user maneuverability may be the air cushion system, which could be powered by the air pump or blower discussed below in relation to the suction that may be needed to raise or otherwise make accessible the desired vessel of the vasculature.

The limb support table 109 preferably includes both a detachable brace 115 and a handgrip 112. The limb support table 109 also preferably includes a separate handgrip support 111 to which the handgrip 112 is affixed. In this manifestation, the handgrip support 111 is preferably slideably attached to the table 109 so that the handgrip support 111 can be slid relative to the main surface 110, as indicated by the double-headed arrow in FIG. 1, thus enabling the table 109 to accommodate limbs of varying size. In lieu of handgrip support 111, the handgrip 112 could be directly affixed to the main surface 110 or other parts of limb support table 109.

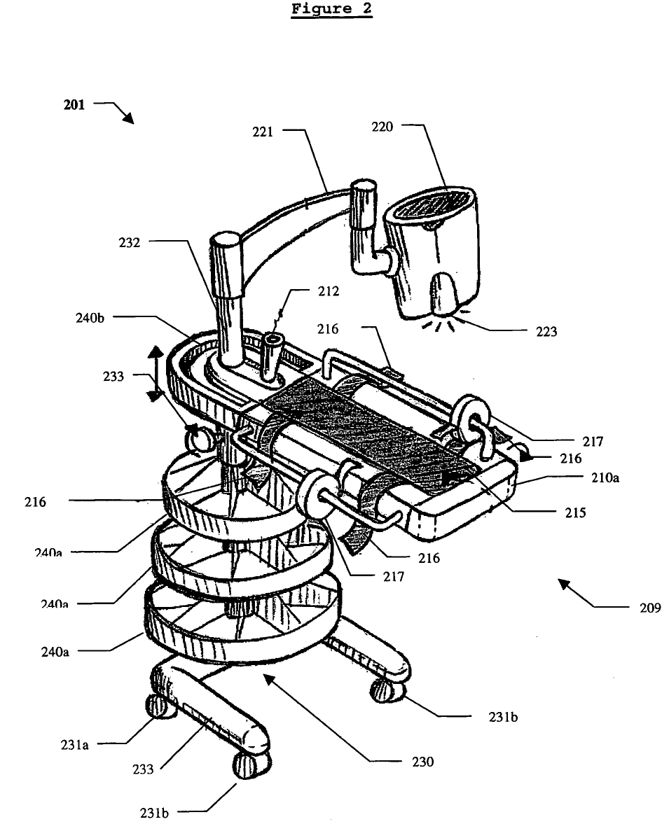

Removably attached to a top surface of limb support table 109, the detachable brace 115 is the component on which the limb is preferably placed. More specifically, a nurse or other qualified personnel would push the IV assist apparatus 101 up to the side of the patient so that the end 110a of table 109 would be nearest to the patient's trunk. The height of limb support table 109 would then be adjusted to a position appropriate for the patient and specifically for the particular limb in which vessel access is to be gained. Although not fully shown in FIG. 1, this adjustment means is akin to that represented by handle 233 in FIG. 2. It could, for example, be manifested as a rotary screw adjustment, a worm gear driven pinion and rack, or an electrically-energized linear actuator. Ideally, the height of the limb support table 109 will be adjusted so that the limb is slightly below the patient's torso, which will cause the desired vessel therein to swell by increasing the pooling of blood therein.

The detachable brace 115 preferably includes one or more straps 116 for securing the limb thereto and thereby limit movement of the limb thereon. The limb should be placed so that the main joint (e.g., elbow or knee) lies in the middle of the brace 115. Because the vast majority of the IV placements are in the forearms, wrists, or hands, only the placement in the antecubital vein will be discussed here in detail. For that reason, the invention herein is primarily described in the context of one limb, namely, the arm. Practitioners skilled in the art will be able, of course, to apply the IV assist apparatus, in all of its embodiments, to other limbs and limb regions.

The straps 116 of brace 115 may take the form of Velcro, adhesive tape, or any other mechanism that secures the arm to the detachable brace 115. With the arm secured to brace 115, the handgrip support 111 can be slid relative to main surface 110 so that the limb support table 109 suitably accommodates the size of the patient's arm. This allows the patient to easily reach and hold the handgrip 112 in his/her hand. Handgrip 112 is preferably made from elastomeric foam so that the patient can squeeze it to increase blood flow to the surface vessels. It is also preferable that handgrip 112 have a non-porous surface for easy cleaning. If some minimal bending of the arm is permissible, merely having the patient hold the handgrip 112 by hand may be sufficient, in which case the detachable brace 115 need not be attached to the arm with straps 116. The arm can then be secured to the limb support table 109 merely by having the patient hold handgrip 112 or simply by the force of gravity or by the nurse or other practitioner holding the arm down on, but not attached to, brace 115.

Unlike prior art devices, the IV assist apparatus 101 of the present invention provides all of the necessary supplies, equipment and a suitable work surface in one mobile, readily accessible, and easy to use apparatus. The supplies and equipment may be stored within the apparatus. In the embodiment of FIG. 1, for example, the storage devices 140 may be manifested as one or more drawers, racks or trays adjustably attached to the structural member 132 of pedestal 130. These storage devices are preferably configured so that they can be moved from side to side so that the user can access them easily from either side. Adjustable dividers (not shown) may be employed to divide the space into compartments or bins of various sizes to store various types of supplies (e.g., needles, catheters, tourniquets, saline test injection syringes) and anything else the user might need. The storage devices 140 could optionally have handles and be liftable off of their supports so that they could be used in different places independent of IV assist apparatus 101. Similarly, the drawers 140 could slide all the way out, for example, for easy transport to a storage cabinet for refilling. Many other drawer, rack and tray arrangements are possible.

Because the storage space is below the limb of the patient, it is important that there be some top or lid to minimize the chance of contamination of any supplies with blood or something else falling from the limb support table 109. One solution would be to use lids that flip up, or just clear rigid sheets that overhang the trays 140. Side openings could be employed so that the users can see into the rack or tray as they reach under the lid for the supplies they need. Drawers have an advantage in that they each slide into an enclosure, the cover of which would preferably be transparent to allow the user to easily view the contents stored therein. One or more of the storage devices 140 or the compartments therein would preferably be used to hold a disposable “sharps container” for easy disposal of the needles and other such disposable instruments. Another of the compartments or storage devices 140 would preferably be used to hold disposable trash containers or bags for non-biohazard waste. Other compartments can be user-selected to hold specific types of materials for recycling.

If the desired vessel can be readily seen, then the needle is inserted therein using customary practices. If the vessel cannot be seen with sufficient confidence, however, there are several recommended techniques to “raise” the vessel. One technique involves placing a warm compress on the limb. A heated pad could be operably associated with limb support table 109, and used when needed to warm the limb. The heated pad would preferably be used with disposable covers. Alternatively, the heated pad (and its disposable covers) could merely be stored within one of the compartments of storage devices 140. It could be pre-warmed and kept warm, be conveniently accessible, and be instantly brought out and applied to the limb whenever needed. Another technique would be to use a heat lamp, either built into vessel visualization device 120 or onto a separate arm (not shown) similar to articulating arm 121. Yet another technique to raise the vessel includes the common practice of gently slapping the limb. This could be replaced or augmented with a continuous or pulsed suction provided by an air pump associated with IV assist apparatus 101. The suction could be conducted to the limb with a flexible tube (not shown). The limb support table 109 could also be lowered to try to better distend the targeted vessel.

The vessel visualization device 120 of the present invention may be used to raise the vessel if none of the aforementioned techniques prove effective or are simply not used. Oftentimes, even if the aforementioned techniques are used, the vessel may be seen only marginally or not at all. As shown in FIG. 1, the vessel visualization device 120 is supported from pedestal 130 via an articulating arm 121. It may also be connected via an articulating arm to the limb support table 109. In either implementation, the vessel visualization device 120 can be moved in several degrees of freedom (as indicated by the arrows in FIG. 1) so that it can placed over and in sufficient proximity to the desired parts of the patient's anatomy.

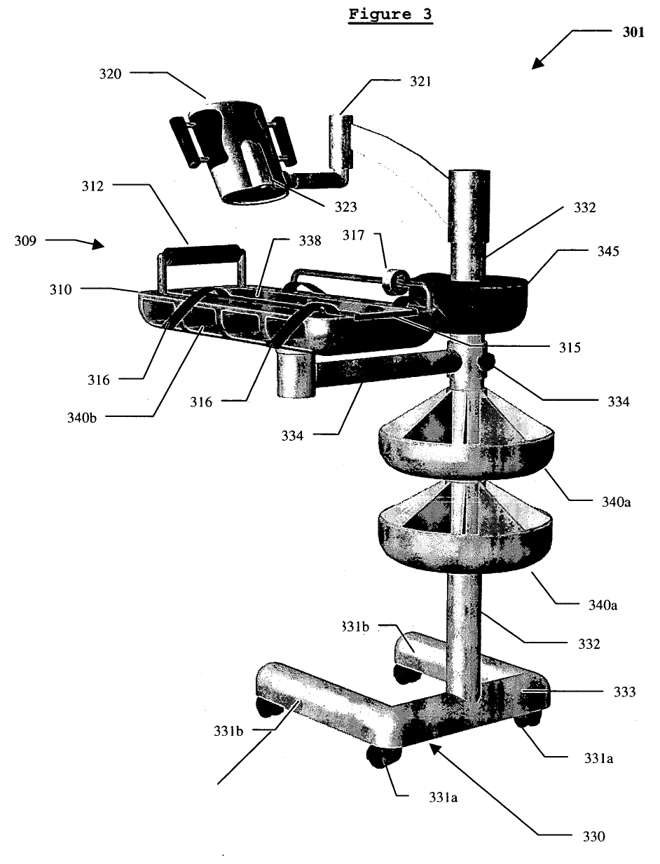

The vessel visualization device 120 can be implemented in a number of ways. A simple magnifying glass could be used to magnify the targeted vessel and the surrounding area. It could also have a spotlight or similar light source to better illuminate the limb. The vessel visualization device 120 could at least in part be a light source, akin to lamp 223 of FIG. 2 and lamp 323 of FIG. 3, that can be brought to the side of the limb. Instead of being articulately connected to pedestal 130 with the magnifying glass, the light source could be connected via a separate articulating arm to limb support table 109. By shining light into the limb at the proper angle, vessels that are somewhat deeper can be seen, with or without a magnifying element. Another alternative is to shine or otherwise position a separable light source into the underside or side of the limb through the detachable brace 115. For this alternative, the detachable brace 115 could have a transparent surface to enable the limb thereon to be backlit therethrough. The vessel visualization device 120 would thus preferably include a shroud to block light from striking the top of the limb. This would help with the visualization of vessels using the side or backlighting techniques.

The vessel visualization device 120 could also be embodied as an infrared sensor and associated liquid crystal display (LCD). In this implementation, the infrared sensor would “illuminate” down onto the limb and the LCD display on top would show the infrared scene below. Infrared light penetrates normal tissues to a greater extent than visible light, as discussed in U.S. Pat. Nos. 4,817,622, 5,519,208, and 6,230,046, the contents of which are incorporated herein by reference. In this particular implementation, the user would effectively look at the limb “through” the infrared imager as they would have done with the magnifying glass, except that the input is infrared light and the output is a visible light image. The user would be able to see both her/his hand and the needle in relation to the patient's limb and vessel. The user would be able to “look through” the vessel visualization device 120 as the needle is inserted into the vessel. Backlighting or side lighting with infrared light is especially useful due to its greater penetration depth.

The vessel visualization device 120 could also be embodied as a small ultrasound imager, either two-dimensional or three-dimensional. If the ultrasound energy is applied from the top, the device 120 could incorporate a needle holder to help the user keep the needle at the proper angle for intersection with and insertion into the vessel. While needle guides are commonly provided with ultrasound equipment, they can also be applied to all the vessel visualization embodiments discussed herein. It is also possible to apply the ultrasound energy from the side or bottom through detachable brace 115. The ultrasound transducer could optionally be connected articulately to limb support table 109. As ultrasound imagers get smaller and smaller, it could be that articulating arm 121 simply holds the unit for ready access. Alternatively, the articulating arm 121 may not be needed at all, in which case the vessel visualization device 120 could be stored in and pulled from one of the compartments when needed.

The vessel visualization device 120 could also be configured so that it is small enough for the practitioner to wear as a “heads up” or “head mounted” display. In this implementation, there could be a different infrared camera and display for each eye. This would enable the practitioner to move her/his head and make sure that the needle is tracking in the correct plane into the vessel. This has the benefit of providing binocular vision, but has the drawback of having to be put on and worn by the practitioner. Between uses it could be stored on articulating arm 121, in one of the storage compartments, or elsewhere.

The vessel visualization device 120 in another implementation could project onto the limb the image seen in infrared or through ultrasound. It could be a black and white or grayscale projection, or it could be a laser-generated diagram of the vessels. The vessel visualization device 120 could also provide a laser guided “flight path” that the operator follows to get the needle into the vessel, similar to the SimpliCT Optical Guidance System currently distributed by Medrad, Inc. of Indianola, Pa.

In a related aspect, a hood may be used with all of the vessel visualization devices 120 disclosed herein. When a practitioner looks into a vessel visualization device, such a hood would reduce the reflected glare from overhead lights, similar to what football referees look into when viewing the instant replay monitor on the field.

Once a suitable vessel has been chosen, it is necessary to insert the needle. Vessels, however, have a tendency to move under the skin. Sometimes the practitioner needs to spread the skin over the vessel or apply pressure to hold the vessel between two fingers so that it doesn't move to the side as the needle is being inserted. The IV assist apparatus 101 could have another articulating arm, similar to articulating arm 121, that has a two pronged or V-notched depressor that traps the vessel and prevents it from moving. Such a vessel immobilizer could also be used to stretch the skin flat. Incorporated into the vessel immobilizer could be a needle guide that helps the practitioner insert the needle at the proper angle into the center of the vessel. Alternatively, the angle guide could be a separate device to help the practitioner maintain the proper angle for optimum insertion. The vessel immobilizer could be articulately connected to either the limb support table 109 or pedestal 130.

A patient typically feels some pain when a needle is inserted into a vein or other vessel. Applying a wipe of local anesthetic to the skin can be useful in this regard. A jet injector may also be used to apply or inject local anesthetic. One type is made by Medi-Ject Corporation; 1840 Berkshire Lane; Minneapolis, Minn. 55441, USA. This has the benefit of injecting local anesthetic painlessly without a needle. Such supplies can be stored in one of the storage devices 140.

As the needle is being inserted into a vessel, it is becomes necessary to stop the movement of the needle quickly before it punctures through the other side of the vessel. Current combined needle/catheter devices use a variety of methods to help indicate when the vessel has been entered. A common one is to have a “flash back” chamber into which blood quickly flows when the needle penetrates the vessel. An alternative is to have some blood simply flow out of the needle and onto a strip of gauze. A better alternative is a Vein Entry Indicator Device (VEID) manufactured by Vascular Technologies of Ness-Ziona, Israel. This device signals very quickly when the pressure therein increases due to the flow of blood from the vessel into the needle. Both of these techniques and others can be used as the practitioner prefers.

Another test for sufficiency of needle placement is to quickly inject 10 ml or so of saline by hand. The saline syringes could be stocked within the storage devices 140 of the IV assist apparatus 101 for ready access.

After confirming the catheter or needle is properly in place, it should be firmly anchored to the skin so that it is not accidentally pulled out or moved, an occurrence which can cause the patient pain and compromise vessel access. If the practitioner wishes, and it was not done previously, the detachable brace 115 can now be attached to the limb to reduce the likelihood that the patient will bend the elbow or other joint and compromise the ability of the vessel to accommodate the injection of fluid.

Disposable covers may be used with detachable brace 115 to assure a clean surface for each subsequent patient or use. More specifically, the brace 115 could be used with a disposable cover, as indicated by numeral 338 in FIG. 3. It is preferable that brace 115 be configured to hold a pack of adhesively-layered disposable covers. After each patient, the top cover is torn off and disposed of, and a clean surface is automatically present for the next patient. Plastic or waxed paper are suitable materials. A small area on which an adhesive similar to that used in Post-It notes would provide sufficient adhesion for each sheet and yet allow easy removal. Alternatively, the brace 115 can be totally disposable, made from cardboard, foam, or thin wood, for example. Another alternative is to have detachable brace 115 be plastic “bubble wrap” similar to what is used for cushioning items for shipping, except that the bubbles are long parallel chambers that, when wrapped around the limb, act similarly to commercial “air splints.” The air pockets would also serve as a cushion for the limb.

Some type of cushioning on limb support table 109 is also preferable for patient comfort. In addition, it would also be preferable that disposable surface-covers be used for limb support table 109. After each patient, the surface-cover would be changed so that each patient contacts a new, clean surface. This saves the practitioner the time of cleaning the surface between patients. As noted above, the detachable brace 115 can be tightened around the limb with Velcro, or it can be attached to the limb with tape. As shown in FIG. 1, the IV assist apparatus 101 preferably includes a tape dispenser 117 operably associated with either the limb support table 109, the pedestal 130, or even other parts of the invention. Regardless of the means used to secure the limb to table 109, it should preferably allow some limited range of motion for comfort and flexibility in positioning for imaging, but not enough motion to potentially compromise the flow of fluid through the vessel. It is also preferable that the practitioner is able to palpate or touch the patient near the end of the needle or IV catheter even if the limb is supported by the detachable brace 115. Palpation at the beginning of an injection is a common practice to detect extravasation.

The detachable brace 115 may optionally serve as a mounting platform for a camera, to watch the injection site and make sure that the injected fluid is traveling properly into the vessel. In this situation, the infrared or visible light camera used in the vessel visualization device 120 could alternatively be attached to the detachable brace 115 so that it moves in a fixed relationship to the patient as the patient moves the limb as directed. Alternatively, the patient's arm could stay attached to the IV assist apparatus 101 during the injection and the vessel visualization device 120 could be used to monitor the limb for possible extravasation. The patient's limb could also be removed from the IV assist apparatus 101 and, after positioning the limb for imaging, the IV assist apparatus 101 can be easily moved so that the vessel visualization device 120 can monitor the site of injection for possible extravasation.

Depending on how it is equipped, the IV assist apparatus 101 may or may not require power. For those configurations of IV assist apparatus 101 that do, the power could be supplied from batteries up in the vessel visualization device 120. Alternatively, the power could be supplied from batteries located near floor level (e.g., near base 133) to lower the center of gravity and increase resistance to tipping. The batteries could be replaced when discharged, recharged by AC power, or recharged by solar cells energized by the room lights. The duty cycle of vessel visualization device 120 could be made pretty low and it could be equipped with an automatic shutoff circuit, which could turn off the device after some predetermined amount of time or if it has not been moved in a certain period of time. Alternatively, the vessel visualization device 120 could receive power through a low or normal voltage cord, preferably from overhead so that it can be easily moved about. Any of these power sources could also be used to operate the other devices mentioned above (e.g., the heating pad or heat lamp, the suction, and/or the air supply for the air cushion system for mobility).

FIG. 2 shows an IV assist apparatus, generally designated 201,according to a first presently preferred embodiment of the invention. It includes a pedestal 230, a mobility means 231, a limb support table 209, and a vessel visualization device 220. It also preferably includes at least one storage device 240. Operably associated with pedestal 230, the mobility means 231 enables the pedestal 230 be mobile. The limb support table 209 is also operably associated with pedestal 230 and is used to support the limb of the patient. The storage device(s) 240 are also operably associated with pedestal 230 and/or the limb support table 209. Used for improving visualization of the vasculature of the patient, the vessel visualization device 220 is operably associated with pedestal 230 and/or the limb support table 209.

As can be seen in the drawings, the embodiments of FIGS. 1 and 2 are largely identical except for a few features. The storage devices 240, for example, preferably take the form of circular trays 240a, which preferably rotate to provide the user with easy access to the supplies stored therein. As with the embodiment of FIG. 1, IV assist apparatus 201 preferably has lids or rigid sheets that overhang its trays 240a. Side openings could be employed so that the users can see into the trays as they reach under the lid for the supplies they need. Adjustable dividers may also be employed to divide trays 240a into compartments or bins of various sizes to store various types of supplies. The compartments and bins may be made removable for easy transport to a storage cabinet for refilling. A designated compartment can be used, of course, to hold the “sharps container.” As with the earlier embodiment, many other drawer, rack and tray arrangements may be used with IV assist apparatus 201.

Another feature introduced with IV assist apparatus 201 is another one of the storage devices 240. More specifically, the limb support table 209 may be equipped with one or more channels 240b or other areas for storage of a small amount of supplies, most likely the particular set of supplies that will be used on the current patient. Yet another feature is handgrip 212, in that it is preferably oriented vertically and near the structural member 232. Handgrip 112 of IV assist apparatus 101, in contrast, is preferably oriented horizontally and opposite of structural member 132. An additional novel feature is found in tape dispenser 217, in that it includes a rail-type member on which the roll(s) of tape may be deployed. In the FIG. 1 embodiment, tape dispenser 117 is preferably of the type that mounts to surface 110 of limb support table 109.

FIG. 3 shows an IV assist apparatus, generally designated 301,according to a second presently preferred embodiment of the invention. It includes a pedestal 330, a mobility means 331, a limb support table 309, and a vessel visualization device 320. It also preferably includes at least one storage device 340. Operably associated with pedestal 330, the mobility means 331 enables the pedestal 330 be mobile. The limb support table 309 is also operably associated with pedestal 330 and is used to support a limb of the patient. Similarly, the storage device(s) 340 are operably associated with pedestal 330 and/or the limb support table 309. Used for improving visualization of the vasculature of the patient, the vessel visualization device 320 is also operably associated with pedestal 330 and/or the limb support table 309.

As can be seen in the drawings, the embodiments of FIGS. 2 and 3 are largely identical except for a few features. One feature introduced with IV assist apparatus 301 is one of the storage devices 340. More specifically, one or more compartments 340b are preferably attached to or built into and/or under limb support table 309. These compartments 340b will thus move along with limb support table 309 whenever it is adjusted by the practitioner via articulating arm 334 in any of several degrees of freedom. The compartments 340b can be used, for example, to store the particular set of supplies that will be used on the current patient. The circular trays 340a are identical to the trays 240a used with IV assist apparatus 201.

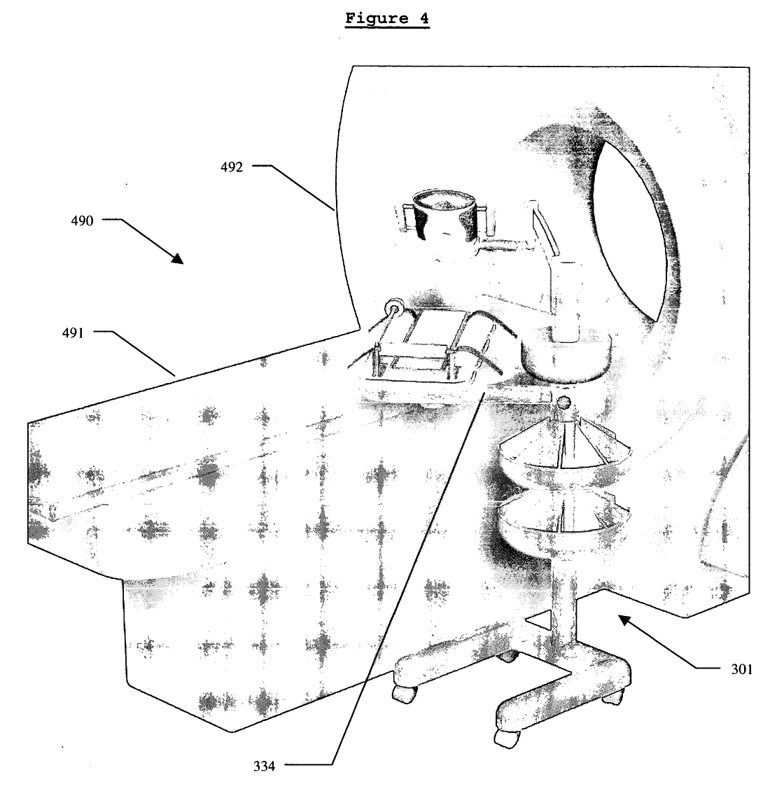

Another feature introduced with this presently preferred embodiment is storage bin 345 situated about structural member 332. It may be used as the sharps container in this embodiment. Handgrip 312 is also manifested differently in this embodiment relative to handgrip 212 of IV assist apparatus 201. FIG. 4 shows the IV assist apparatus 301 next to the patient table 491 and scanner 492 of imager 490.

In the embodiments of FIGS. 1-3, the limb support tables of IV assist apparatuses 101, 201 and 301 are mounted on pedestals 130, 230 and 330, respectively. Each of those pedestals preferably includes a base 133/233/333 and a single vertically-disposed structural member 132/232/332. Of course, many other mounting systems besides the above-described pedestals are possible. For example, a pole in the middle is one option, as are two or more poles. Persons skilled in the art can implement the pedestals 130, 230 and 330 in a variety of ways depending upon the specific imagers, room geometries, and practitioner actions that are to be accommodated. FIG. 4 shows the IV assist apparatus 301 of FIG. 3 rolled into one possible position adjacent to imager 490. The imager 490 has a patient table 491 (sometimes called a couch or bed) and a scanner 492. Imagers 490 manufactured by different companies, of course, have different dimensions for their patient tables 491 and scanners 492.

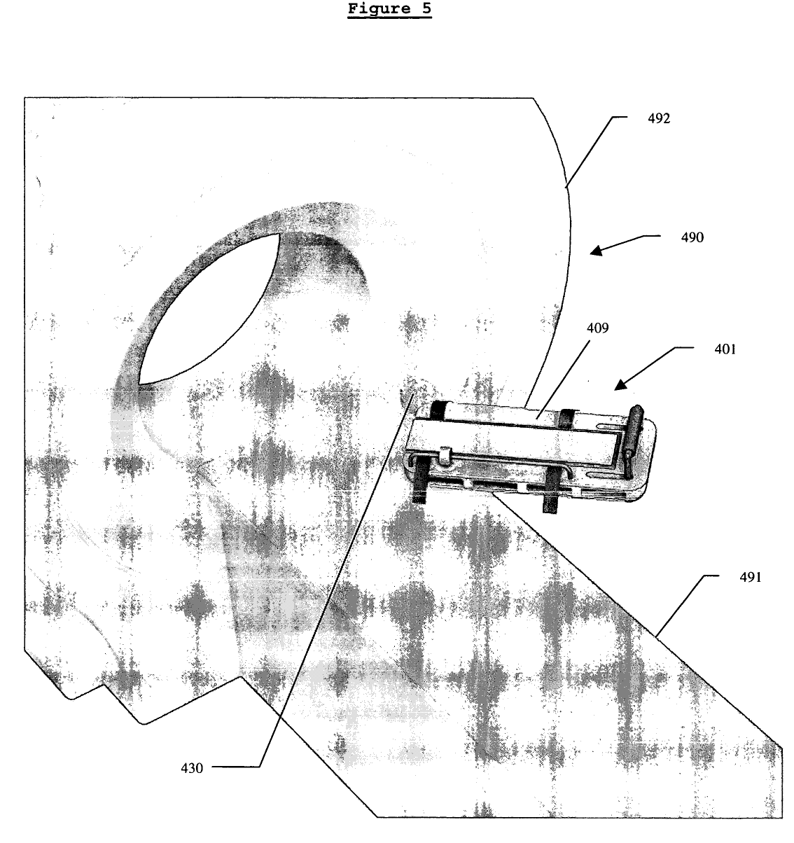

FIG. 5 shows an IV assist apparatus, generally designated 401,according to a third presently preferred embodiment of the invention. This embodiment can be particularly well adapted to any imaging suite regardless of its dimensions or the sizes of its various components. IV assist apparatus 401 preferably includes a limb support table 409, a mounting system 430, and a vessel visualization device as described above. The mounting system 430 is used to mount the limb support table 409 and the vessel visualization device in proximity to and adjustably with respect to the patient.

The mounting system 430 of IV assist apparatus 401 can be implemented in a number of ways. Preferably, it can be manifested as an articulating arm by which limb support table 409 is mounted to the side of patient table 491 of imager 490, as is shown in FIG. 5. It can also be implemented as a overhead counterpoise so that the IV assist apparatus 401 can be moved up and out of the way when not in use. For use outside of an imaging suite, for example, it can take the form of an articulating arm by which limb support table 409 is mounted to a separate chair or recliner for use, for example, in a dedicated IV access station. In this way, IV assist apparatus 401 could be integrated with drug delivery pumps or injectors, or with blood withdrawal systems such as plasma or platelet phoresis or dialysis machines.

As with the embodiments of FIGS. 2 and 3, the limb support table 409 may be equipped with one or more channels or built-in compartments for storage of a small amount of supplies. For all of the IV assist apparatuses disclosed herein, such storage areas provide the significant benefit to the user of having all the necessary supplies readily available. Rather than acquiring supplies from storage bins on a distant counter, the storage devices make the supplies readily available right near the patient.

The IV assist apparatuses of the present invention will enable medical practitioners to quickly and efficiently gain IV access in even difficult patients, and they will reduce the likelihood that such vessel access will be compromised if the patient has to reposition the relevant limb during an imaging procedure.

While the preferred and alternative embodiments have been described primarily in relation to use during an imaging procedure, it should be understood that the invention disclosed herein can be used in all situations where IV access is needed, e.g., in the arms, legs or other parts of the body. Similarly, many types of nurses, phlebotomists, or other medical practitioners may need to achieve vessel access and thus could benefit from using the IV assist apparatuses and method claimed below.

The presently preferred and alternative embodiments for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations which fall within the literal meaning, and range of equivalency, of the claims are to be embraced within their scope. Persons of such skill will also recognize that the scope of the invention is indicated by the following claims rather than by any particular example or embodiment discussed in the foregoing description.

What is claimed is:

1. An apparatus for use in accessing a vasculature of a patient, the apparatus comprising:
(a) a pedestal;
(b) mobility means operably associated with said pedestal for enabling said pedestal to be mobile;
(c) a limb support table operably associated with said pedestal for supporting a limb of the patient;
(d) at least one storage device operably associated with at least one of said pedestal and said limb support table; and
(e) a vessel visualization device operably associated with at least one of said pedestal and said limb support table for improving visualization of the vasculature of the patient.

2. The apparatus of claim 1 wherein said pedestal comprises:
(a) a base; and
(b) a structural member, attached to said base, to which said limb support table is adjustably securable.

3. The apparatus of claim 1 wherein said mobility means comprises a plurality of at least one of wheels and glides attached to said pedestal.

4. The apparatus of claim 1 wherein said mobility means comprises an air cushion system attached to said pedestal.

5. The apparatus of claim 1 wherein said limb support table comprises:
(a) a brace detachably attachable to a top surface of said limb support table for contact with the limb of the patient; and
(b) a handgrip for the patient to grip.

6. The apparatus of claim 5 wherein said brace comprises at least one strap for securing the limb to said brace and thereby limit movement thereof.

7. The apparatus of claim 5 wherein said brace is capable of being used with a disposable cover therefor.

8. The apparatus of claim 5 wherein said brace is disposable.

9. The apparatus of claim 5 wherein said brace has a transparent surface for enabling the limb to be backlit therethrough.

10. The apparatus of claim 5 wherein said limb support table further comprises a handgrip support to which said handgrip is affixed, said handgrip support being slideably attached to said limb support table so that said limb support table can accommodate the patient essentially regardless of a size of the limb thereof.

11. The apparatus of claim 1 further comprising a brace detachably attached to said limb support table for contact with the limb of the patient.

12. The apparatus of claim 11 wherein said brace comprises at least one strap for securing the limb to said brace and thereby limit movement thereof.

13. The apparatus of claim 11 wherein said brace is capable of being used with a disposable cover therefor.

14. The apparatus of claim 11 wherein said brace is disposable.

15. The apparatus of claim 11 wherein said brace has a transparent surface for enabling the limb to be backlit therethrough.

16. The apparatus of claim 1 further comprising:
(a) a handgrip for the patient to grip; and
(a) a handgrip support to which said handgrip is affixed, said handgrip support being slideably attached to said limb support table so that said limb support table can accommodate the patient essentially regardless of a size of the limb thereof.

17. The apparatus of claim 1 further comprising a heated pad associated with said limb support table for warming the limb.

18. The apparatus of claim 1 wherein said least one storage device comprises a compartment at least one of incorporated underneath and attached to said limb support table.

19. The apparatus of claim 1 wherein said least one storage device comprises a channel defined within said limb support table.

20. The apparatus of claim 1 wherein said least one storage device comprises a tray adjustably attached to said pedestal.

21. The apparatus of claim 1 wherein said least one storage device comprises a drawer adjustably attached to said pedestal.

22. The apparatus of claim 21 wherein said drawer includes at least one of compartments and bins of various sizes.

23. The apparatus of claim 1 wherein said least one storage device comprises a rack adjustably attached to said pedestal.

24. The apparatus of claim 23 wherein said rack includes at least one of compartments and bins of various sizes.

25. The apparatus of claim 1 wherein said vessel visualization device includes a magnifying glass connected articulately to at least one of said pedestal and said limb support table.

26. The apparatus of claim 1 wherein said vessel visualization device includes a light connected articulately to at least one of said pedestal and said limb support table.

27. The apparatus of claim 1 wherein said vessel visualization device includes an infrared imager connected articulately to at least one of said pedestal and said limb support table.

28. The apparatus of claim 1 wherein said vessel visualization device includes an ultrasound imager connected articulately to at least one of said pedestal and said limb support table.

29. The apparatus of claim 1 wherein said vessel visualization device includes an optically-guided system for guiding and positioning of a needle for puncture into a desired vessel of the vasculature.

30. The apparatus of claim 1 further comprising an adjustment means for enabling a position of said limb support table to be adjustable relative to said pedestal.

31. The apparatus of claim 1 further comprising at least one strap for securing the limb to said limb support table and thereby limit movement thereof.

32. The apparatus of claim 1 further comprising a power supply for providing electrical power to at least said vessel visualization device.

33. An apparatus for use in accessing a vasculature of a patient, the apparatus comprising:
(a) a limb support table for supporting a limb of the patient;
(b) a vessel visualization device for improving visualization of the vasculature of the patient; and
(c) a mounting system for mounting said limb support table and said vessel visualization device in proximity to and adjustably with respect to the patient.

34. The apparatus of claim 33 wherein said limb support table comprises:
(a) a brace detachably attachable to a top surface of said limb support table for contact with the limb of the patient; and
(b) a handgrip for the patient to grip.

35. The apparatus of claim 34 wherein said brace comprises at least one strap for securing the limb to said brace and thereby limit movement thereof.

36. The apparatus of claim 34 wherein said brace is capable of being used with a disposable cover therefor.

37. The apparatus of claim 34 wherein said brace is disposable.

38. The apparatus of claim 34 wherein said brace has a transparent surface for enabling the limb to be backlit therethrough.

39. The apparatus of claim 34 wherein said limb support table further comprises a handgrip support to which said handgrip is affixed, said handgrip support being slideably attached to said limb support table so that said limb support table can accommodate the patient essentially regardless of a size of the limb thereof.

40. The apparatus of claim 33 further comprising a brace detachably attached to said limb support table for contact with the limb of the patient.

41. The apparatus of claim 40 wherein said brace comprises at least one strap for securing the limb to said brace and thereby limit movement thereof.

42. The apparatus of claim 40 wherein said brace is capable of being used with a disposable cover therefor.

43. The apparatus of claim 40 wherein said brace is disposable.

44. The apparatus of claim 40 wherein said brace has a transparent surface for enabling the limb to be backlit therethrough.

45. The apparatus of claim 33 further comprising:
(a) a handgrip for the patient to grip; and
(a) a handgrip support to which said handgrip is affixed, said handgrip support being slideably attached to said limb support table so that said limb support table can accommodate the patient essentially regardless of a size of the limb thereof.

46. The apparatus of claim 33 further comprising a heated pad associated with said limb support table for warming the limb.

47. The apparatus of claim 33 wherein said mounting system comprises an overhead counterpoise by which said limb support table is mountable in proximity to the patient.

48. The apparatus of claim 33 wherein said mounting system includes a first articulating arm by which said limb support table is mountable to a table for the patient.

49. The apparatus of claim 33 wherein said mounting system includes a first articulating arm by which said limb support table is mountable to a separate chair for the patient.

50. The apparatus of claim 33 wherein said mounting system includes a mobile pedestal to which said limb support table is adjustably mounted and thus made mobile.

51. The apparatus of claim 33 wherein said vessel visualization device is connected articulately to said limb support table via a second articulating arm.

52. The apparatus of claim 33 wherein said vessel visualization device includes a magnifying glass connected articulately to said limb support table.

53. The apparatus of claim 33 wherein said vessel visualization device includes a light connected articulately to said limb support table.

54. The apparatus of claim 33 wherein said vessel visualization device includes an infrared imager connected articulately to said limb support table.

55. The apparatus of claim 33 wherein said vessel visualization device includes an ultrasound imager connected articulately to said limb support table.

56. The apparatus of claim 33 wherein said vessel visualization device is wearable over the eyes by an operator-thereof.

57. The apparatus of claim 33 wherein said vessel visualization device includes an optically-guided system for guiding and proper positioning of a needle for puncture into a desired vessel of the vasculature.

58. The apparatus of claim 33 further comprising a storage device attached to or incorporated into at least one of said limb support table and said mounting system.

59. The apparatus of claim 58 wherein said storage device comprises a channel defined within said limb support table.

60. The apparatus of claim 58 wherein said storage device comprises a drawer slideable within said limb support table.

61. The apparatus of claim 60 wherein said drawer includes at least one of compartments and bins of various sizes.

62. The apparatus of claim 33 further comprising a vessel immobilizer device operably associated with at least one of said limb support table and said mounting system.

63. The apparatus of claim 33 further comprising a heat lamp operably associated with at least one of said limb support table and said mounting system.

64. The apparatus,of claim 33 further comprising an air pump for at least providing suction to raise a vessel of the vasculature.

65. The apparatus of claim 33 further comprising a camera operably associated with at least one of said limb support table and said mounting system for close monitoring of the limb and the vasculature thereof.

66. The apparatus of claim 33 further comprising a tape dispenser operably associated with at least one of said limb support table and said mounting system.

67. The apparatus of claim 33 further comprising a power supply for providing electrical power to the apparatus.

68. A method of accessing a vasculature of a patient, the method comprising the steps of:
(a) providing the apparatus of claim A1;
(b) moving the apparatus in proximity to the patient;
(c) placing a limb of the patient on said limb support table;
(d) retrieving an access conduit from said at least one storage device;
(e) inserting said access conduit into a vessel of the patient;
(f) strain relieving said access conduit; and
(g) removing the limb of the patient from said limb support table.

69. The method of claim 68 further comprising the step of using said vessel visualization device to improve visualization of the vessel before accessing the vessel with said access conduit.

70. The method of claim 68 further comprising the step of securing the limb of the patient to said limb support table.

71. The method of claim 68 further comprising the step of adjusting the position of said limb support table.

72. The method of claim 68 further comprising the step of moving the apparatus away from the patient.
